(19) United States
(12) Patent Application Publication
(10) Pub. No.: US 2024/0058382 A1
(43) Pub. Date: Feb. 22, 2024

(54) DETERMINISTIC MECHANOPORATION FOR CELL ENGINEERING

(71) Applicants: The Regents of the University of California, Oakland, CA (US); City of Hope, Duarte, CA (US)

(72) Inventors: Masaru P. Rao, Riverside, CA (US); Harish G. Dixit, San Jose, CA (US); Hideaki Tsutsui, Riverside, CA (US); Morgan L. Dundon, Riverside, CA (US); Pranee I. Pairs, Riverside, CA (US); Stephen J. Forman, Duarte, CA (US); Christine E. Brown, Duarte, CA (US); Renate Starr, Duarte, CA (US); Christopher B. Ballas, Palmyra, NJ (US)

(21) Appl. No.: 18/497,798

(22) Filed: Oct. 30, 2023

Related U.S. Application Data
(63) Continuation of application No. 16/814,768, filed on Mar. 10, 2020, now abandoned.
(60) Provisional application No. 62/816,831, filed on Mar. 11, 2019.
(60) Provisional application No. 62/837,067, filed on Apr. 22, 2019.

(51) Int. Cl. A61K 35/17; C07K 14/725; C12N 15/11; C12N 15/87; C12N 5/0783

(52) U.S. Cl. CPC A61K 35/17; C07K 14/7051; C12N 15/111; C12N 15/87; C12N 5/0638; A61K 9/0019

(57) ABSTRACT

Intracellular delivery of a genetic construct to immune cells including: obtaining a deterministic mechanoporation (DMP) platform that includes a substrate having a surface and a plurality of capture sites, each said capture site having a boundary shape at the surface adapted and configured to support thereon a cell, and each said capture site having a bottom and including a sub-micron-scale projection extending from the bottom toward the surface of the substrate, wherein said projection is adapted and configured to penetrate a cell membrane and/or wall of the cell, and wherein the substrate has a plurality of aspiration vias situated at the bottom of the capture sites; introducing the cells to the surface in a liquid media; capturing the cells within the capture sites by applying a first hydrodynamic force; applying a second hydrodynamic force on the captured cell and locally rupturing the membrane and/or wall of the cell with the projection, introducing the genetic construct into the cells, and releasing the porated cells from the capture sites. Also disclosed are methods of chimeric antigen receptor (CAR) T cell adoptive immunotherapy and T cell receptor (TCR) therapy.

### STATEMENT REGARDING FEDERALLY SPONSORED RESEARCH AND DEVELOPMENT

This invention was made with government support under Grant Nos. R21 RR026253 and R21GM0103973, awarded by the National Institutes of Health. The government has certain rights in the invention.

### BACKGROUND

The safe and efficient introduction of exogenous materials into large populations of suspension cells is a key requisite for a growing number of applications based on engineered cell products. Notable examples include ex vivo cell therapies for the treatment of hematologic disorders and malignancies, wherein hematopoietic stem cells or T lymphocytes are modified outside the body to replace, correct, or add targeted genes, after which they are infused into the patient to perform their intended function (e.g., reconstitute dysfunctional cell lineages, augment stem cell transplantation, or redirect immune response to fight cancer, infection, or autoimmunity) (Naldini, L. Nat Rev Genet 2011, 12(5), 301-315; Scott, C. T. and DeFrancesco, L., Nat Biotechnol 2016, 34(6), 600-607; Aldoss, I. et al. Leukemia 2017, 31(4), 777-787; Sadelain, M. et al. Nature 2017, 545(7655), 423-431; June, C. H. et al. Science 2018, 359(6382), 1361-1365; and Dunbar, C. E. et al. Science 2018, 359 (6372)). While viral transduction has been the most common method used for genetic manipulation in these applications, concerns such as insertional mutagenesis and scalability of vector production, among others, have driven interest in the development of non-viral transfection methods (Qasim, W. et al. Drugs 2014, 74(9), 963-969; Wang, X. et al. Cancer Gene Ther 2015, 22(2), 85-94; Roh, K.-H. et al. Annual Review of Chemical and Biomolecular Engineering 2016, 7(1), 455-478; and Roth, T. L. et al. Nature 2018, 559(7714), 405-409) Similarly, these and other limitations, such as viral packaging and cargo constraints, have more broadly motivated the development of intracellular delivery techniques for a wide range of other applications in biology, medicine, and cellular biomanufacturing (Meacham, J. M. et al. J Lab Autom 2014, 19(1), 1-18; Stewart, M. P. et al. Nature 2016, 538(7624), 183-192; Marx, V. Nat Methods 2015, 13(1), 37-40; and Stewart, M. P. et al. Chem Rev 2018, 118(16), 7409-7531).

Emerging microfluidic approaches for achieving intracellular delivery via physical disruption (i.e., poration) of the plasma membrane have shown promise for addressing many of these limitations, and within the context of cells in suspension specifically, recent examples include inertial microfluidic cell hydroporation (iMCH) (Deng, Y. et al. Nano Lett 2018, 18(4), 2705-2710), squeeze cell poration (SQZP) (Sharei, A. et al. Proceedings of the National Academy of Sciences 2013, 110(6), 2082-2087; and Sharei, A. et al. PLoS One 2015, 10(4), e0118803), acoustic shear poration (ASP) (Zarnitsyn, V. G. et al. Biomed Microdevices 2008, 10(2), 299-308; and Meacham, J. M. et al. Sci Rep 2018, 8(1), 3727) and nanochannel electroporation (NEP) (Boukany, P. E. et al. Nat Nanotechnol 2011, 6(11), 747-754; and Chang, L. et al. Lab Chip 2015, 15(15), 3147-3153). However, while most have shown potential for scaling to the throughputs required for engineered cell product manufacturing (i.e., millions to billions of cells), many are nevertheless subject to tradeoffs between delivery efficiency and cellular viability. This is particularly the case for the delivery of larger cargos (e.g., genetic constructs) to difficult-to-transfect cells that are often of interest for therapeutic applications (e.g., primary, immune, & stem cells) (Gresch, O. and Altrogge, L., In Protein Expression in Mammalian Cells: Methods and Protocols, Hartley, J. L., Ed. Humana Press: Totowa, N J, 2012; pp 65-74; Zhao, Y. et al. Mol Ther 2006, 13(1), 151-159; and Lakshmipathy, U. et al. Stem Cells 2004, 531-543). One potential cause for this tradeoff may lie in the inherent stochasticity of the poration process in most of these approaches. When coupled with the need to produce pores of sufficient size to enable efficient uptake of large cargos, this may result in the formation of a multitude of large pores that ultimately compromises viability. The physiochemical impacts of these “uncontrolled” methods likely contribute to the difficulties in achieving efficient and viable transfection. In contrast, one of the distinguishing features of mechanoporation (DMP) is the deterministic introduction of a pore. The stochastic nature of other approaches produces a delivery distribution curve, with some cells getting none, other cells getting a large amount of delivered material, and everything in-between. Advantageously, mechanoporation (DMP) produces a more uniform distribution. Since both high efficiency and high viability are crucial requirements for engineered cell product manufacturing in general, and therapeutic applications in particular (Aijaz, A. et al. Nature Biomedical Engineering 2018, 2(6), 362-376), need remains for the development of intracellular delivery techniques that are scalable, efficient, and able to preserve viability.

### SUMMARY

Some embodiments relate to a method of intracellular delivery of a genetic construct to immune cells including:
obtaining a deterministic mechanoporation (DMP) platform that includes a substrate having a surface and a plurality of capture sites, each said capture site having a boundary shape at the surface adapted and configured to support thereon a cell, and each said capture site having a bottom and including a sub-micron-scale projection extending from the bottom toward the surface of the substrate, wherein said projection is adapted and configured to penetrate a cell membrane and/or wall of the cell, and wherein the substrate has a plurality of aspiration vias situated at the bottom of the capture sites;
introducing the cells to the surface in a liquid media;
capturing the cells within the capture sites by applying a first hydrodynamic force;
applying a second hydrodynamic force on the captured cell and locally rupturing the membrane and/or wall of the cell with the projection,
introducing the genetic construct into the cells, and
releasing the porated cells from the capture sites.

In some examples, the immune cells are T cells.

In some examples, the T cells are primary human T cells.

In some examples, a mean transfection yield of the immune cells is from 20-100%.

In some examples, a transfection yield of the immune cells is 2 fold to 20-fold higher than a transfection yield obtainable by bulk electroporation.

In some examples, the genetic construct encodes a chimeric antigen receptor (CAR) that recognizes a specific antigen, wherein the CAR comprises an extracellular antigen recognition domain, a transmembrane domain and a cytoplasmic signaling domain that stimulates the immune cells to target and attack cells expressing the specific antigen.

In some examples, the genetic construct encodes a T cell receptor (TCR) that recognizes a specific peptide displayed in the context of MHC II molecules, wherein the TCR is involved in a pathway that stimulates immune cells to target and attack cells expressing an antigen that contains the specific peptide.

In some examples, the substrate of the DMP platform has greater than 10⁶ capture sites.

In some examples, a flow rate of the liquid media containing the cells is adjusted to a first flow rate during capture to achieve a capture site occupancy of at least 50%.

In some examples, a second flow rate of the liquid media containing the cells during application of the second hydrodynamic force is different compared to the first flow rate during capture.

In some examples, a mean transfection yield of the cells is between 50-100%.

In some examples, a mean transfection yield of the cells is between 75-100%.

In some examples, a mean transfection yield of the cells is between 90-100%.

In some examples, the projection has a length comparable to the radius of the cells.

In some examples, the genetic construct is introduced into the nucleus of the cells.

Some embodiments relate to a method of chimeric antigen receptor (CAR) T cell adoptive immunotherapy in a subject including:
obtaining T cells transfected with a chimeric antigen receptor (CAR) gene by the method of claim 1, wherein the CAR recognizes a cell surface antigen of a tumor cell, and administering the cells to the subject.

In some examples, the subject is treated for a disease selected from the group consisting of a cancer, an infection, human immunodeficiency virus (HIV) infection, transplant rejection and autoimmunity.

In some examples, the subject is treated for a cancer.

In some examples, the cancer is any non-blood or non-hematopoietic based cancer or any solid tumor forming or infiltrating/metastatic cancer, for example a cancer selected from the group consisting of a bone cancer, an endocrine cancer, a germ cell cancer, a kidney cancer, a liver cancer, a neuroblastoma and a soft tissue cancer.

In some examples, the T cells are autologous to the patient.

In some examples, the autologous T cells are administered intravenously as a bolus dose.

In some examples, the T cells are allogenic to the patient.

In some examples, the allogenic T cells are administered intravenously as a bolus dose.

Some embodiments relate to a method of T cell receptor (TCR) therapy in a subject including:
obtaining T cells transfected with a T cell receptor gene by the method of claim 1, wherein the T cell receptor recognizes a specific peptide displayed in the context of MHC II molecules on antigen presenting cells, and
administering the cells to the subject.

In some examples, the subject is treated for a disease selected from the group consisting of a cancer, an infection, human immunodeficiency virus (HIV) infection, transplant rejection and autoimmunity.

### BRIEF DESCRIPTION OF THE DRAWINGS

FIG. 1. Schematic diagram of CAR T Cell Therapy. A DNA containing a CAR gene is delivered to a cell and the cell expresses the CAR gene to produce the CAR protein.

FIG. 2. Schematic diagram of Engineered T Cell Receptor (TCR) Therapy. A DNA containing a TCR gene is delivered to a cell and the cell expresses the TCR gene to produce the TCR protein.

FIG. 3. Deterministic mechanoporation (DMP). (a) Concept, illustrated for a single Capture Site. Cells are captured using negative Aspiration flow, porated by impingement upon the Penetrator, and released by reversal of flow, after which intracellular delivery occurs via diffusive influx of exogenous cargo through the single transient plasma membrane pore. (b) Design schematics with quarter section removed in isometric views to allow visualization of key device features. Actual devices contain a 100×100 array of Capture Sites. (c) Fabrication process. Silicon-on-insulator substrates are coated with front and backside SiO₂ layers, which are then patterned and used as masks for dry etching. (d) Scanning electron micrograph of a portion of the device array, with inset showing a higher magnification image of a single Capture Site (scale bar=5 μm). (e) Schematic illustrating the packaging of the device chip, its placement upon the stage of a fluorescence microscope, and its connection to a programmable syringe pump for fluidic actuation of the aspiration circuit. The inset shows a photograph of the packaged device on the microscope stage.

FIG. 4. Capture optimization study, with Jurkats serving as a model suspension cell line. (a, b) Representative fluorescence micrographs of portions of the DMP device array showing high Capture Site occupancy after capture at 30 μL/min, and lower occupancy at 60 μL/min, respectively. The images share identical magnification (Scale bar=100 μm). (c) Plot of capture efficiency as a function of capture flow rate. Highest efficiency (71%) was observed at 30 μL/min (*: p≤0.05; **: p≤0.01; ***: p≤0.001; -: no statistical significance). Data=mean t standard deviation (n=3).

FIG. 5. Puncture optimization study using Jurkats, with propidium iodide (PI) serving as a model, membrane-impermeable, small-molecule exogenous cargo, and Cell Tracker Green (CTG) serving as a post-DMP cellular viability marker. (a) Representative flow cytometry data for cells subjected to 40 μL/min puncture flow rate. Gates 1, 2, and 3 encompass the population of intact cells (i.e., events with size and granularity consistent with intact cells), viable intact cells (i.e., CTG+), and viable intact cells with exogenous cargo delivered (i.e., both CTG+ & PI+), respectively. (b) Plots of cellular viability, delivery efficiency, and delivery yield as a function of puncture flow rate (*: p≤0.05; **: p≤0.01; ***: p≤0.001; -: no statistical significance). High delivery efficiencies were seen for all conditions, with highest efficiency at 40 μL/min (93%). Gating was established using the control data presented in FIG. 7, which also showed that there was minimal passive uptake of the PI cargo, and negligible autofluorescence in the spectral ranges of interest. Data=mean t standard deviation (n=3).

FIG. 6. DMP validation study using Jurkat, K-562, and primary human T cells, with GFP plasmid serving as a model genetic construct cargo, and Calcein Blue AM (CBAM) serving as a post-incubation cellular viability marker. (a, b, c) Representative flow cytometry data for DMP-based transfection of Jurkat, K-562, and primary human T cells, respectively. Gates 1, 2, and 3 encompass the population of intact cells, viable intact cells (i.e., CBAM+), and viable intact cells with delivery and expression of the plasmid cargo (i.e., both CBAM+ & GFP+), respectively. (d) Plots of cellular viability, transfection efficiency, and transfection yield for DMP vs. conventional bulk electroporation (BEP) for Jurkat (JRKT), K-562 (K562), and primary human T cells (PRIM) (*: p≤0.05; **: p≤0.01; ***: p≤0.001; -: no statistical significance). High viability, efficiency, and yield were observed for DMP-transfected Jurkats (all >87%), with mean yield over four times that of BEP (88% vs. 20%, respectively). Efficient DMP-based transfection of K-562 and primary human T cells was also observed, albeit with lower yield than the Jurkats (49% and 82%, respectively, vs. 88% for Jurkats). Gating was established using the control data presented in FIG. 8, which also showed that there was minimal passive uptake and expression of the plasmid cargo, and negligible autofluorescence in the spectral ranges of interest. Representative flow cytometry data and controls for the BEP benchmarking studies are presented in FIGS. 9-11. Data=mean t standard deviation (n=3).

FIG. 7. Representative flow cytometry control data used to establish the cascading gating scheme for the puncture optimization study (i.e., FIG. 5), with Jurkats serving as a model suspension cell line, propidium iodide (PI) serving as a model, membrane-impermeable, small-molecule exogenous cargo, and Cell Tracker Green (CTG) serving as a post-DMP cellular viability marker. (a) Non-porated cell control. The cells were subjected to the same pre- and post-DMP protocols used for the puncture optimization samples. However, rather than running the cells through the DMP device, they were instead incubated in PBS/glycerol for 7 min to simulate total time in the device. Gates 1, 2, and 3 encompass the population of intact cells (i.e., events with size and granularity consistent with intact cells), viable intact cells (i.e., CTG+), and viable intact cells with passive uptake of the exogenous cargo (i.e., both CTG+ & PI+), respectively. The high proportion of cells in Gate 2, and low proportion in Gate 3, indicated good viability and minimal passive uptake of PI, respectively, in the non-porated cells. (b) Autofluorescence control. The cells were not subjected to any staining, nor poration. Instead, they were simply incubated in PBS/glycerol for 7 min to simulate total time in the DMP device, followed immediately by incubation in PBS for another 30 min to simulate the total time of the post-DMP staining used for the puncture optimization samples. Gates 1, 2, and 3 encompass the population of intact cells, intact CTG+ cells, and intact CTG+ & PI+ cells, respectively. The low proportions of cells in Gates 2 and 3 indicated that there was negligible autofluorescence in these spectral ranges.

FIG. 8. Representative control data used to establish the cascading gating scheme for the DMP validation study (i.e., FIG. 6), using Jurkat, K-562, and primary human T cells, with GFP plasmid serving as a model genetic construct cargo, and Calcein Blue AM (CBAM) serving as a post-incubation cellular viability marker. (a, b, c) Non-porated cell controls for Jurkat, K-562, and primary human T cells, respectively. The cells were subjected to the same pre- and post-DMP protocols used for the DMP validation samples. However, rather than running the cells through the DMP device, they were instead incubated in PBS/glycerol for 7 min to simulate total time in the device. Gates 1, 2, and 3 encompass the population of intact cells, viable intact cells (i.e., CBAM+), and viable intact cells with passive uptake and expression of the plasmid cargo (i.e., both CBAM+ & GFP+), respectively. The high proportion of cells in Gate 2, and low proportion in Gate 3, indicated good viability and minimal passive uptake and expression of the plasmid, respectively, in the non-porated cells. (d, e, f) Autofluorescence controls for Jurkat, K-562, and primary human T cells, respectively. The cells were not subjected to any staining, plasmid exposure, or poration. Instead, they were simply incubated in PBS/glycerol for 7 min to simulate total time in the DMP device, followed immediately by incubation in PBS for another 30 min to simulate the total time of the post-DMP plasmid incubation, and finally, followed by the same 12 h incubation protocol used for the DMP validation samples. Gates 1, 2, and 3 encompass the population of intact cells, intact CBAM+ cells, and intact CBAM+ & GFP+ cells, respectively. The low proportions of cells in Gates 2 and 3 indicated that there was negligible autofluorescence in these spectral ranges.

FIG. 9. Representative Jurkat BEP benchmarking data and controls for the DMP validation study (i.e., FIG. 6), with GFP plasmid serving as the cargo, and DAPI serving as a post-incubation cellular viability marker. (a) Representative flow cytometry data for BEP cells. Gates 1, 2, and 3 encompass the population of intact cells, viable intact cells (i.e., DAPI−), and viable intact cells with delivery and expression of the plasmid cargo (i.e., both DAPI− & GFP+), respectively. (b) Non-porated cell control. The cells were subjected to the same pre- and post-BEP protocols used for the BEP samples. However, rather than running the cells through the BEP instrument, they were instead incubated in fresh media with GFP for 7 min to simulate total time in the instrument. Gates 1, 2, and 3 encompass the population of intact cells, viable intact cells (i.e., DAPI−), and viable intact cells with passive uptake and expression of the plasmid cargo (i.e., both DAPI− & GFP+), respectively. The high proportion of cells in Gate 2, and low proportion in Gate 3, indicated good viability and minimal passive uptake and expression of the plasmid cargo, respectively, in the non-porated cells. (c) Autofluorescence control. The cells were not subjected to any staining, plasmid exposure, or poration. Instead, they were simply incubated in fresh media for 1 min to simulate total time in the BEP instrument, followed by the same 12 h incubation protocol used for the BEP samples. Gates 1, 2, and 3 encompass the population of intact cells, intact DAPI− cells, and intact DAPI− & GFP+ cells, respectively. The low proportion of cells outside of Gate 2, and the low proportion within Gate 3 indicated that there was negligible autofluorescence in these spectral ranges.

FIG. 10. Representative K-562 BEP benchmarking data and controls for the DMP validation study (i.e., FIG. 6), with GFP plasmid serving as the cargo, and DAPI serving as a post-incubation cellular viability marker. (a) Representative flow cytometry data for BEP cells. Gates 1, 2, and 3 encompass the population of intact cells, viable intact cells (i.e., DAPI−), and viable intact cells with delivery and expression of the plasmid cargo (i.e., both DAPI− & GFP+), respectively. (b) Non-porated cell control. The cells were subjected to the same pre- and post-BEP protocols used for the BEP samples. However, rather than running the cells through the BEP instrument, they were instead incubated in fresh media with GFP for 7 min to simulate total time in the instrument. Gates 1, 2, and 3 encompass the population of intact cells, viable intact cells (i.e., DAPI−), and viable intact cells with passive uptake and expression of the plasmid cargo (i.e., both DAPI− & GFP+), respectively. The high proportion of cells in Gate 2, and low proportion in Gate 3, indicated good viability and minimal passive uptake and expression of the plasmid cargo, respectively, in the non-porated cells. (c) Autofluorescence control. The cells were not subjected to any staining, plasmid exposure, or poration. Instead, they were simply incubated in fresh media for 1 min to simulate total time in the BEP instrument, followed by the same 12 h incubation protocol used for the BEP samples. Gates 1, 2, and 3 encompass the population of intact cells, intact DAPI− cells, and intact DAPI− & GFP+ cells, respectively. The low proportion of cells outside of Gate 2, and the low proportion within Gate 3 indicated that there was negligible autofluorescence in these spectral ranges.

FIG. 11. Representative primary human T cell BEP benchmarking data and controls for the DMP validation study (i.e., FIG. 6), with GFP plasmid serving as the cargo, and DAPI serving as a post-incubation cellular viability marker. (a) Representative flow cytometry data for BEP cells. Gates 1, 2, and 3 encompass the population of intact cells, viable intact cells (i.e., DAPI−), and viable intact cells with delivery and expression of the plasmid cargo (i.e., both DAPI− & GFP+), respectively. (b) Non-porated cell control. The cells were subjected to the same pre- and post-BEP protocols used for the BEP samples. However, rather than running the cells through the BEP instrument, they were instead incubated in fresh media with GFP for 7 min to simulate total time in the instrument. Gates 1, 2, and 3 encompass the population of intact cells, viable intact cells (i.e., DAPI−), and viable intact cells with passive uptake and expression of the plasmid cargo (i.e., both DAPI− & GFP+), respectively. The high proportion of cells in Gate 2, and low proportion in Gate 3, indicated good viability and minimal passive uptake and expression of the plasmid cargo, respectively, in the non-porated cells. (c) Autofluorescence control. The cells were not subjected to any staining, plasmid exposure, or poration. Instead, they were simply incubated in fresh media for 1 min to simulate total time in the BEP instrument, followed by the same 12 h incubation protocol used for the BEP samples. Gates 1, 2, and 3 encompass the population of intact cells, intact DAPI− cells, and intact DAPI− & GFP+ cells, respectively. The low proportion of cells outside of Gate 2, and the low proportion within Gate 3 indicated that there was negligible autofluorescence in these spectral ranges.

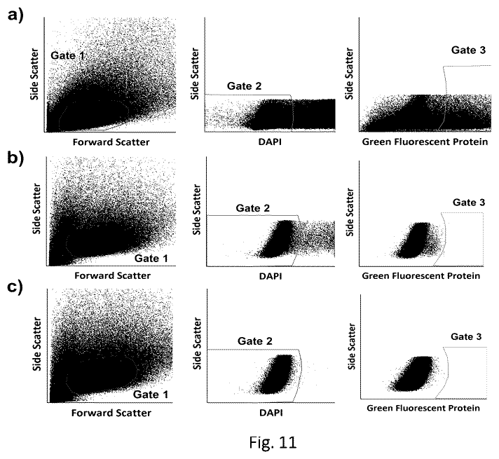

### DESCRIPTION

We disclose the use of a microfluidic device for transfecting suspended cells that is specifically designed to meet the needs of engineered cell product manufacturing. The methods include for deterministic mechanoporation (DMP) of large numbers of cells, each at a single site in their plasma membrane, and doing so in a manner that allows rapid collection of the cells for subsequent processing. We show that DMP enables efficient delivery of large-molecule cargos while minimizing damage to the cell, thus allowing achievement of transfection yields that exceed both conventional and emerging non-viral transfection techniques. DMP provides a new means for addressing critical roadblocks in the development and manufacture of ex vivo cell therapies based on engineered T cells (e.g., adoptive cancer immunotherapies based on chimeric antigen receptor modified T cells or engineered T cell receptor (TCR) therapies).

The deterministic mechanoporation platform (DMP) to transfect cells results in surprising and unexpected transfection yields, e.g., a mean transfection yield as high as 88%, compared to a mean transfection yield of only 20% for bulk electroporation (BEP). For instance, we demonstrate transfection yields for primary human T cells of 82% using DMP, compared to only 20% using BEP. These high transfection yields could not have been predicted based on our previous disclosure of an ultrahigh throughput microinjection device (U.S. Pat. No. 9,885,059 B2).

The deterministic mechanoporation platform exceeds the performance of other known microfluidic intracellular platforms, particularly with regard to T cells (e.g., Jurkat cells) and other cells in general (Deng, Y.; Kizer, M.; Rada, M.; Sage, J.; Wang, X.; Cheon, D. J.; Chung, A. J., Intracellular Delivery of Nanomaterials Via an Inertial Microfluidic Cell Hydroporator. Nano Lett 2018, 18 (4), 2705-2710; Chang, L.; Gallego-Perez, D.; Zhao, X.; Bertani, P.; Yang, Z.; Chiang, C. L.; Malkoc, V.; Shi, J.; Sen, C. K.; Odonnell, L., et al., Dielectrophoresis-Assisted 3d Nanoelectroporation for Non-Viral Cell Transfection in Adoptive Immunotherapy. Lab Chip 2015, 15 (15), 3147-3153, Chang, L.; Bertani, P.; Gallego-Perez, D.; Yang, Z.; Chen, F.; Chiang, C.; Malkoc, V.; Kuang, T.; Gao, K.; Lee, L. J., et al., 3d Nanochannel Electroporation for High-Throughput Cell Transfection with High Uniformity and Dosage Control. Nanoscale 2016, 8 (1), 243-252; Chang, L.; Gallego-Perez, D.; Chiang, C.-L.; Bertani, P.; Kuang, T.; Sheng, Y.; Chen, F.; Chen, Z.; Shi, J.; Yang, H., et al., Controllable Large-Scale Transfection of Primary Mammalian Cardiomyocytes on a Nanochannel Array Platform. Small 2016, 12 (43), 5971-5980; and Ding, X.; Stewart, M.; Sharei, A.; Weaver, J. C.; Langer, R. S.; Jensen, K. F., High-Throughput Nuclear Delivery and Rapid Expression of DNA Via Mechanical and Electrical Cell-Membrane Disruption. Nat Biomed Eng 2017, 1). The unexpected results are particularly relevant to immune cells, which are known to be refractory to transfection.

In some examples, use of the DMP platform results a mean transfection yield of from 20-100%. Transfection yields achieved can be 20%, 25%, 30%, 35%, 40%, 45%, 50%, 55%, 60%, 65%, 70%, 75%, 80%, 85%, 90%, 95% or 100%.

In some examples, the transfection yield obtained using the DMP platform can be 2-fold to 20-fold higher than a transfection yield obtainable by bulk electroporation. For example, the transfection yield obtained using the DMP platform can be 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold or 20-fold higher than a transfection yield obtainable by bulk electroporation.

Other advantages of using the deterministic mechanoporation platform to transfect cells include the ability to effect intra-nuclear delivery, as opposed to cytosolic delivery with sometimes inefficient nuclear uptake, and avoidance of side effects specific to viral transduction and chemical transfection techniques. Disclosed are methods of intracellular delivery of genetic constructs using the deterministic mechanoporation platform. Specific embodiments relate to transfection of immune cells, specifically immune T cells transfected with chimeric antigen receptor (CAR T), and related CAR T therapies, including solid tumor treatments. Other specific embodiments relate to transfection of T cells for the purpose of T cell receptor (TCR) therapy.

The DMP platform opens a singular pore in the plasma membrane that serves as pathway for diffusive transport of constructs into the cell.

In DMP, the constructs are not injected into the nucleus. Instead DMP opens a hole in the outer plasma membrane and the nuclear membrane, thus providing a path for transport of the constructs into the cytoplasm and nucleus.

In some embodiments, the DMP platform is used to transfect primary human T cells, which are relevant for use in CAR-T therapies or Engineered T cell Receptor (TCR) therapies, for example.

In some autologous therapies, cells are delivered by a single administration or by only a few administrations. A typical dosage is hundreds of millions of cells to a few billion cells. The cells may be delivered as a single infusion, multiple infusion, or a bolus dose intravenously (IV). Generally, infusion takes about 30-90 minutes. For cancers forming solid tumors and cancers that spread out and do not form tumors, infusion route may vary significantly. For example, cells many may directly injected into a tumor or a region of a cancer, while other means of administration are into cavities or spaces near the tumor or region of a cancer. In some cases, there may be multiple injection sites, for example around the brain, etc. The method of creating the cellular product need only be sufficient to produce a number of modified cells to meet the dose requirement, regardless of delivery approach. In some embodiments, autologous T cells are administered on average in amounts of 1×10⁷, 1×10⁸, 2×10⁸, 3×10⁸, 4×10⁸, 5×10⁸, 6×10⁸, 7×10⁸, 8×10⁸, 9×10⁸, 10×10⁸, 1×10⁹, 1.5×10⁹, 2×10⁹, 2.5×10⁹, 3×10⁹, 3.5×10⁹, 4×10⁹, 4.5×10⁹, or 5×10⁹, 5.5×10⁹ cells per kg.

The number of allogenic cells administered is typically higher than for autologous therapies. In some embodiments, allogenic T cells are administered on average in amounts of 5×10⁸, 6×10⁸, 7×10⁸, 8×10⁸, 9×10⁸, 10×10⁸, 1×10⁹, 1.5×10⁹, 2×10⁹, 2.5×10⁹, 3×10⁹, 3.5×10⁹, 4×10⁹, 4.5×10⁹, or 5×10⁹, 5.5×10⁹ cells per kg.

Using deterministic mechanoporation (DMP), large numbers of cells can be transfected, with mean transfection yields between 50-100%. In some cases, the mean transfection yield of the cells, e.g., T cells, is between 75-100%, 90-100% or above 95%.

### CAR-T Cell Therapy

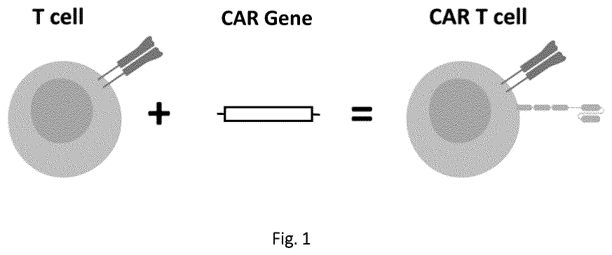

In CAR-T cell therapy, a patient's T cells are changed in the laboratory so they will attack cancer cells. T cells are taken from a patient's blood. Using CAR T cell therapy, a patient's T cells are equipped with a synthetic receptor known as a CAR, which stands for chimeric antigen receptor (FIG. 1). A key advantage of CARs is their ability to bind to cancer cells even if their antigens are not presented on the surface via MHC, which can render more cancer cells vulnerable to their attacks. However, CAR T cells can only recognize antigens that themselves are naturally expressed on the cell surface. Thus, the range of potential antigen targets is smaller than with TCRs. In October 2017, the U.S. Food and Drug Administration (FDA) approved the first CAR T cell therapy to treat adults with certain types of large B-cell lymphoma.

Given their power, CARs are being explored in a variety of strategies for many cancer types. One approach currently in clinical trials is using stem cells to create a limitless source of off-the-shelf CAR T cells. This may allow doctors to treat patients in an expedited fashion.

CAR-T applications beyond cancer include treatment of infection (e.g., HIV) and undesired immune response (e.g., autoimmunity & transplant rejection). As reviewed in Maldini et al. “CAR T cells for infection, autoimmunity and allotransplantation” Nat Rev Immunol 18(10): 605-616, chimeric antigen receptors (CARs) have shown remarkable ability to re-direct T cells to target CD19-expressing cells, e.g., CD19 expressing blood cancer cells in leukemia and lymphoma, resulting in remission rates of up to 90% of individuals with pediatric acute lymphoblastic lymphoma. Lessons learned from these clinical trials of adoptive T cell therapy for cancer, as well as investments made in manufacturing T cells at commercial scale, provide a basis for additional applications using CARs. This technology may be used to target infectious diseases such as HIV and undesired immune responses such as autoimmunity and transplant rejection.

### Engineered T Cell Receptor (TCR) Therapies

Engineered T cell Receptor (TCR) therapies may be used to address limitations of CAR-T in cancer indications, including solid tumor forming cancers via TIL (tumor infiltrating lymphocyte) based therapy. Unfortunately, not all patients have T cells that have already recognized the cancer cells. Other patients might, but for a number of reasons, these T cells may not be capable of being activated and expanded to sufficient numbers to enable rejection of cancer cells. For these patients, doctors may employ an approach known as engineered T cell receptor (TCR) therapy.

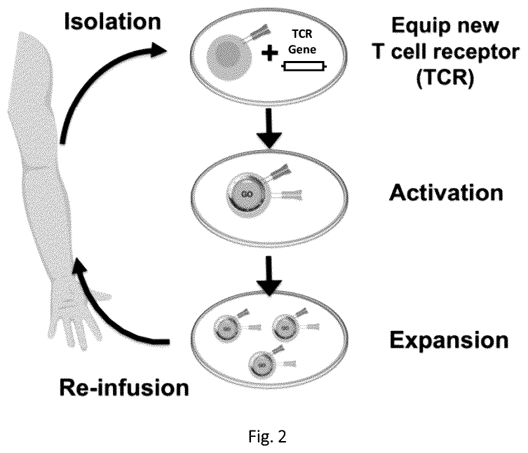

This approach also involves taking T cells from patients, but instead of just activating and expanding the available anti-tumor T cells, the T cells can also be equipped with a new T cell receptor that enables them to target specific cancer antigens (FIG. 2). By allowing doctors to choose an optimal target for each patient's tumor and distinct types of T cell to engineer, the treatment can be further personalized to individuals and, ideally, provide patients with greater hope for relief.

In some examples, a genetic construct encodes a T cell receptor (TCR) that targets a specific antigen, wherein the TCR stimulates immune cells to target and attack cells expressing the specific antigen.

CAR-T cell therapies and Engineered T cell Receptor (TCR) therapies may involve additional elements included together or individually in a genetic construct. For example, additional expression constructs may contain cytokines, growth factors, pathway influencing or modulating factors (like PD-1/PDL-1 pathway blocking constructs), etc. In addition, constructs may be used to gene edit, or change the nature of the cells (for differentiation purposes like converting active cells to memory cells, or effector cells) or to make allogeneic products.

Many of these technologies, especially the allogeneic variety, will include transfection with multiple constructs and/or more complex constructs than simply delivering a CAR transgene or a TCR transgene. This may include immune function modifying factors like cytokines, dominant negative inhibitors (e.g. dnPD-1), trafficking chemokines (CXCRs), editing constructs like CRISPR or TALEN, etc.

### Adoptive Cell Transfer

Adoptive Cell Transfer is a process of harvesting and subsequent introduction or re-introduction of cells into a patient. The process can use patient cells (autologous) or cells from a donor (allogenic). The cells can be genetically engineered. Manufacturing engineered cell products for adoptive T cell cancer immunotherapy “trains” a patient's immune system to recognize and eradicate cancerous cells and tumors. Efficacy is dependent on tumor-specific antigen recognition and other factors such as tumor microenvironment, co-factors delivered with therapy, immunosuppression pathways (PD-1/PDL-1) expressed, etc. In some cases, dose level will also play a role.

CAR (Chimeric Antigen Receptor) Engineering introduces novel receptors for cancer marker recognition into a T-cell and provides a basis for treating a majority of cancer histologies.

Transduction is typically defined as a viral and/or permanent delivery, whereas transfection is generally defined as non-viral and/or non-permanent delivery, primarily of a nucleic acid. Prior strategies for transducing genes encoding tumor specificity into T-Cells via intracellular delivery include: (a) DNA electro-transfer, which has the limitation of insufficient antitumor efficacy due to extended culture times, and (b) viral transduction, which has the limitations of poor scalability, cost-complexity for large scale manufacturing, and DNA size dependence, which result in a bottleneck for phase 3 trials and beyond.

Mechanoporation provides a means for achieving intracellular delivery with greater: (a) precision that results in improved expression from optimized delivery, (b) uniformity, which results in reduced ex vivo expansion time and increased antitumor efficacy, and (c) cargo versatility (e.g., co-expression, multiple transfections).

The number of cells needed or to be produced will be based on dose requirement on a per kg basis. The dose is dependent upon the technology specific to the developer of the therapy (one CAR T therapy might use 10⁷ cells/kg another might use 10⁹ cells/kg), the indication (including whether pediatric or adult), and to some degree, perhaps, autologous vs allogenic. The dose is generated after a period of expansion following the transfection/transduction (depending on the technology and what is delivered). The delivery is typically accomplished early in the manufacturing process utilizing a typical number of cells generated from the leukopheresis. Scalability of the DMP device design easily meets these needs. For laboratory-scale, a 100 mm wafer provides >10 million sites/device and a 150 mm wafer provides >30 million sites/device. For commercial-scale, a 200 mm wafer provides >60 million sites/device and a 300 mm wafer provides >120 million sites/device.

Using the DMP platform, the number of transfected cells produced per day can be between 10⁶-10¹². For example, a number of transfected autologous cells or allogenic cells produced per day can be about 10⁶, 10⁷, 10⁸, 10⁹, 10¹⁰, 10¹¹, or 10¹².

Chimeric antigen receptor (CAR) T cell cancer immunotherapy is a revolutionary treatment that primes a patient's own immune system to better recognize and fight tumors. The first two CAR T therapies were approved in 2017, both for blood cancers.

Poor scalability of DNA electro-transfer and viral transduction preclude large-scale manufacturing. While CAR T is the most promising technology in oncology today, limitations related to the use of electroporation and viral vectors involve inefficiency of delivery, safety concerns, viability, and limited ability to optimize/control delivery.

### Example 1

### Massively-Parallelized. Deterministic Mechanoporation for Intracellular Delivery

Microfluidic intracellular delivery approaches based on plasma membrane poration have shown promise for addressing the limitations of conventional cellular engineering techniques in a wide range of applications in biology and medicine. However, the inherent stochasticity of the poration process in many of these approaches often results in a trade-off between delivery efficiency and cellular viability, thus potentially limiting their utility. Herein, we present a novel microfluidic device concept that mitigates this trade-off by providing opportunity for deterministic mechanoporation (DMP) of cells en masse. This is achieved by the impingement of each cell upon a single needle-like Penetrator during aspiration-based capture, followed by diffusive influx of exogenous cargo through the resulting single membrane pore, once the cells are released by reversal of flow. Massive parallelization enables high throughput operation, while single-site poration allows for delivery of small and large-molecule cargos in difficult-to-transfect cells with efficiencies and viabilities that exceed both conventional and emerging transfection techniques. As such, DMP shows promise for advancing cellular engineering practice in general, and engineered cell product manufacturing in particular.

We have previously reported early results from efforts focused on addressing this need via the development of a novel microfluidic device concept that enables high throughput intracellular delivery in suspension cells through deterministic mechanoporation (DMP) (Zhang, Y. et al. IEEE, 2012; pp 594-597). Herein, we expand upon this initial report by detailing results from more recent efforts focused on optimization and validation of the DMP concept. We demonstrate significant improvement in small-molecule delivery performance relative to our earlier efforts. We also report the first validation of this concept within the context of large-molecule delivery (i.e., GFP plasmid), where we observe high expression efficiency and cellular viability, thus leading to transfection yields that exceed a commercially-optimized bulk electroporation protocol by over four-fold. Finally, we demonstrate the versatility of this technique through efficient transfection of human cell lines and primary cells of relevance to ex vivo cell therapies. Collectively, these results illustrate the promise embodied in DMP for addressing critical roadblocks in the development and manufacture of engineered cell products.

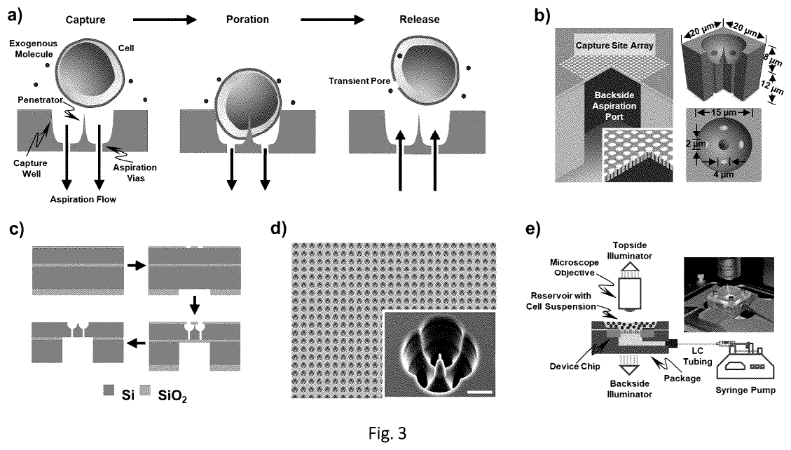

As illustrated in FIG. 3 (a), the DMP concept relies upon a unique device architecture consisting of a large array of Capture Sites, each composed of a hemispherical Capture Well with a single, sub-μm-scale, needle-like Penetrator projecting from the bottom of the well, as well as a multiplicity of Aspiration Vias situated at the bottom of the well. Together, these features enable intracellular delivery on a single-cell basis, but massively-parallelized scale, via the capture and release of cells en masse by aspiration flow, followed by diffusive influx of exogenous cargo through the transient plasma membrane pore produced within each cell by their impingement upon the Penetrator. In doing so, this provides opportunity for achieving deterministic poration at a single site in the plasma membrane, for each cell within a large population. This therefore enables minimization of cellular damage, and thus, offers potential for maximizing both efficiency and viability, unlike stochastic shear-based mechanoporation approaches (e.g., SQZP, ASP, & iMCH). Furthermore, unlike other penetration-based mechanoporation techniques (Shalek, A. K. et al. Proc Natl Acad Sci USA 2010, 107 (5), 1870-1875; Peer, E. et al. ACS Nano 2012, 6 (6), 4940-4946; Xie, X. et al. ACS Nano 2013, 7(5), 4351-4358; Wang, Y. et al. Nat Commun 2014, 5, 4466-4474; Peng, J. et al. ACS Nano 2014, 8 (5), 4621-4629; Chiappini, C. et al. Nat Mater 2015, 14 (5), 532-539; and Elnathan, R. et al. Advanced Functional Materials 2015, 25 (46), 7215-7225) the coupling of deterministic poration with aspiration-based cell manipulation provides a more facile means for rapidly and efficiently manipulating large populations of suspension cells, and importantly, collecting them immediately afterwards for subsequent processing (e.g., expansion, culture/fermentation, cryopreservation, transplantation, etc.).

FIG. 3, b illustrates the DMP device design, which is defined within a silicon-on-insulator substrate using conventional microfabrication processes. Key features include: a) sizing of the Capture Wells for the intended cell type; b) use of multiple Aspiration Vias within each Capture Well, to uniformly tension the plasma membrane during capture, and thus, facilitate penetration; c) use of Penetrators with sub-μm tip diameters, to minimize penetration force, and thus, stress upon the cells; d) direct connection of all Aspiration Vias to a large common Backside Aspiration Port, to ensure uniform flow across the array; and e) minimized unit cell size, which enables high-density arraying of the Capture Sites (e.g., 2500 sites/mm², with total array size of 10⁴ sites in the current study). Taken together, these design features simplify operation and impart intrinsic scalability, thus providing promise for meeting the needs of many engineered cell product manufacturing applications. For example, scaling to greater than 10⁷ Capture Sites would be easily possible within the current 100 mm diameter substrates, thus enabling transfection of sufficient numbers of cells for autologous cancer immunotherapies based on the adoptive transfer of chimeric antigen receptor (CAR) modified T cells (Wang, X. et al. Journal of Immunotherapy 2012, 35 (9), 689-701). Similarly, further scaling to greater than 10⁸ Capture Sites would be possible within larger 300 mm substrates, thus enabling throughputs approaching those required for future allogeneic CAR T therapies, as well as a wide range of cellular biomanufacturing applications (e.g., production of therapeutic proteins, antibodies, viral vectors, etc.).

The device fabrication process, presented in FIG. 3 (c), was designed to enable definition of all Capture Site features using a single frontside mask, to simplify fabrication and further ensure scalability. Using a mask pattern consisting solely of four elliptical Aspiration Vias per Capture Site, a combination of isotropic and anisotropic reactive ion etching steps was employed to define the Capture Site Array over the large Backside Aspiration Port. Scanning electron micrographs of a completed device (FIG. 3, d) demonstrate the realization of a uniform array of Capture Sites, each containing a single Penetrator with sub-μm tip diameter. However, Capture Well geometry is observed to deviate slightly from the intended hemispherical profile, due to transport limitations during the isotropic etching step. FIG. 3, e illustrates the DMP device packaging, which was designed for placement on the stage of a fluorescence microscope. The package provides an open reservoir above the chip for introduction and collection of cells, as well as a port beneath the chip for fluidic communication with a programmable syringe pump for bidirectional actuation of the aspiration circuit (i.e., withdrawal mode to produce negative aspiration flow through the device for cell capture and poration, and infuse mode to produce positive aspiration flow for cell release).

In our initial report on the DMP device concept (Zhang, Y. et al. IEEE, 2012; pp 594-597) low delivery efficiencies were observed for small-molecule cargos (˜15%), and subsequent investigation suggested that poor cell capture efficiency was one potential cause. As such, new studies were initiated to better understand the effect of capture flow rate on capture efficiency, and inform optimization thereof. In these new studies, an immortalized human T lymphocyte cell line (Jurkat) was selected for use, due to its relevance for ex vivo cell therapies (e.g., as a model for the study & development of CAR T therapies). In order to facilitate visualization during device operation, the cells were first labelled using a membrane-permeable viability stain, Calcein Blue AM (CBAM), which is enzymatically-cleaved after entry into the cytosol, thus resulting in the formation of a fluorescent dye product that is retained within cells with intact plasma membranes. The cells were then introduced into the device and subjected to varying capture flow rates, followed by manual pipetting to wash uncaptured cells from the array and remove them from the reservoir. Finally, a mosaic of fluorescence images that encompassed the entirety of the device array was collected, and capture efficiency was determined using image analysis software.

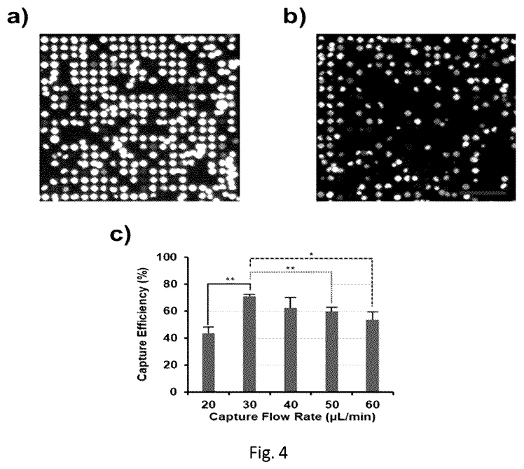

These studies showed that high Capture Site occupancy (71%) could be achieved at flow rates of 30 μL/min (FIG. 4, a). However, markedly lower occupancy was observed at higher flow rates (FIG. 4, b), which suggested that many of the cells in the unoccupied sites had been lysed. This was corroborated by the diminished fluorescence intensity and non-spherical morphologies seen for many of the captured cells at the higher flow rates (FIG. 4, b), which may have been caused by partial lysis and efflux of fluorescent CBAM molecules from the cytosol. The reduced capture efficiency observed at the lowest flow rate (FIG. 4, c) indicated that this too was disadvantageous, presumably because it was insufficient to retain the cells during washing. This therefore established 30 μL/min as the optimal capture flow rate for use in the remainder of the studies reported herein.

Due to the viscoelastic nature of the plasma membrane, the device operation cycle also included a negative aspiration flow pulse after the capture step to facilitate puncture, thus necessitating optimization of this parameter as well. In these studies, the Jurkats were first CBAM-stained (for visualization), and then introduced into the device and subjected to capture at the optimal 30 μL/min flow rate. Propidium iodide (PI) was also included in the device reservoir, and the cells were subjected to varying puncture flow rates after capture, followed by the removal of the uncaptured cells from the device. While PI is typically used to quantify dead cells, in the current study it served as a model, membrane-impermeable, small-molecule exogenous cargo (668 Da) that allowed for fluorescence-based confirmation of delivery (upon intercalation with the cellular DNA). After the puncture and wash steps, the aspiration flow was reversed to release the captured cells, which were then collected and co-incubated with PI and CellTracker Green (CTG) for 30 min, the former of which continued to serve as a cargo molecule, and the latter of which served as a post-DMP cellular viability marker (via retention of the enzymatically-cleaved fluorescent dye product). Finally, the cells were centrifuged and resuspended for flow cytometry.

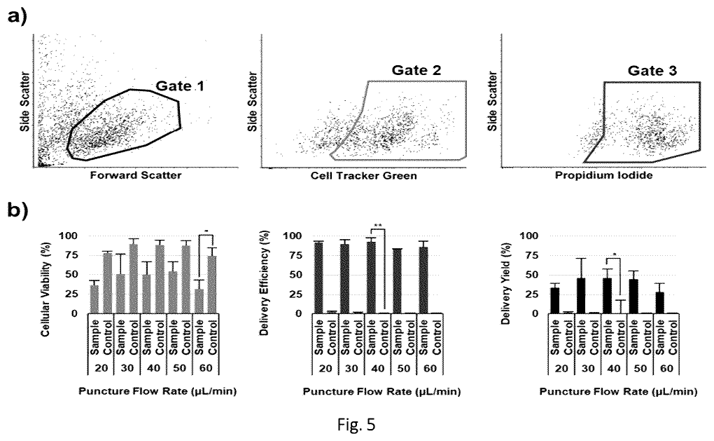

FIG. 5 shows representative flow cytometry data from these studies, as well as plots summarizing the effect of puncture flow rate on cellular viability (i.e., the percentage of viable cells amongst the population of intact cells recovered from the device), delivery efficiency (i.e., the percentage of cells with delivered PI cargo amongst the population of viable intact cells), and delivery yield (i.e. the percentage of viable cells with PI cargo delivered amongst the population of intact cells, which is equivalent to the product of the cellular viability and delivery efficiency). It is important to note that, while commonplace, quantification in this manner does not provide means for evaluating cell losses due to the device or subsequent processing (e.g., cells lost to lysis, adhesion to cultureware surfaces, incomplete pelleting/resuspension, etc.). High delivery efficiencies were observed for all puncture flow rates, reaching as high as 93% at 40 μL/min, thus establishing this as the optimal puncture flow rate for the Jurkats. Importantly, this also represented a six-fold improvement in small-molecule delivery performance relative to our initial reports (Zhang, Y. et al. IEEE, 2012; pp 594-597). While more modest cellular viabilities (and thus delivery yields) were observed, we hypothesize that this may have been an artifact resulting from the persistence of the plasma membrane pore, or transient enhancement of membrane permeability more globally, both of which would allow efflux of the fluorescent CTG products from the cytosol. However, further studies are required to confirm this conjecture, particularly since plasma membrane repair and resealing is typically expected within a few seconds to a few minutes after mechanical injury (McNeil, P. L. et al. The Journal of Cell Biology 1997, 137 (1), 1-4; McNeil, P. L. Journal of Cell Science 2002,115 (5), 873-879; and Andrews, N. W. et al. Trends Cell Biol 2014, 24 (12), 734-742).

With the operational parameters optimized, we proceeded to validation of the DMP device concept within the context of large-molecule delivery. In these studies, a reporter DNA construct was included in the reservoir (GFP plasmid, 4.7 kbp), and the Jurkats were subjected to the optimal capture and puncture flow rates established earlier (i.e., 30 μL/min & 40 μL/min, respectively), followed by removal of the uncaptured cells. Afterwards, the captured cells were released and collected, incubated with the plasmid for 30 min, centrifuged, resuspended in fresh media, and incubated for 12 h. The cells were then centrifuged, stained with CBAM to evaluate post-incubation viability, followed by centrifugation and resuspension for flow cytometry. To evaluate the versatility of DMP concept, the Jurkat-optimized protocol was also used to transfect an immortalized human myelogenous leukemia cell line (K-562) with the GFP plasmid. Similar to the Jurkats, the K-562 cells were selected due to their relevance for ex vivo cell therapies (e.g., as artificial antigen presenting cells for mediation of CAR T cell expansion ex vivo (Butler, M. O. et al. Immunological Reviews 2014, 257 (1), 191-209; and Rushworth, D. et al. Journal of Immunotherapy 2014, 37 (4), 204-213) or control targets for evaluation of CAR T cell product potency in vitro (Wang, X. et al. Journal of Immunotherapy 2012, 35(9), 689-701; and Tumaini, B. et al. Cytotherapy 2013, 15 (11), 1406-1415). Finally, to demonstrate the potential for clinical relevance, primary human T cells were also transfected using the Jurkat-optimized DMP protocol. For the purposes of benchmarking, separate sets of all tested cell types were subjected to conventional bulk electroporation (BEP) using manufacturer-optimized protocols for each cell type, and GFP plasmid concentrations consistent with both the manufacturer recommendations, and the DMP validation studies (i.e., 20 μg/mL for all cell types).

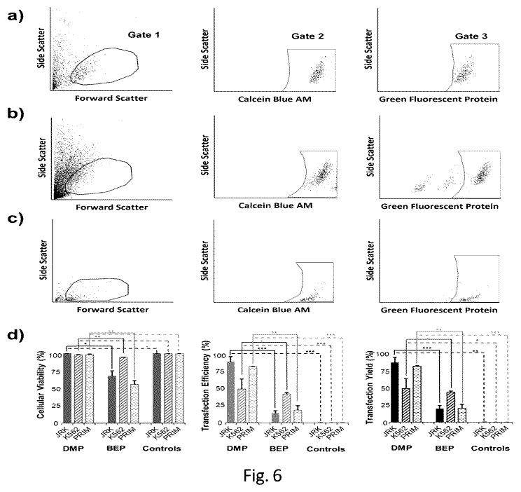

FIG. 6 presents representative flow cytometry data from these studies, as well plots comparing cellular viability, transfection efficiency, and transfection yield for DMP versus BEP for all cell types. Relatively low viability and efficiency were observed for the BEP-based Jurkat transfection, thus leading to a mean transfection yield of 20%, an unsurprising result given that T cells are notoriously refractory to most conventional non-viral transfection techniques.41 Conversely, excellent viability and efficiency were observed for DMP-based Jurkat transfection, thus resulting in a mean transfection yield of 88%, an over four-fold improvement relative to the BEP benchmark. Importantly, this also exceeds the performance reported for other microfluidic intracellular delivery platforms for delivery of comparable GFP reporter plasmids to Jurkats specifically (Meacham, J. M. et al. Sci Rep 2018, 8 (1), 3727; and Boukany, P. E. et al. Nat Nanotechnol 2011, 6(11), 747-754) as well as other cell types more generally (Deng, Y. et al. Nano Lett 2018, 18(4), 2705-2710; Chang, L. et al. Lab Chip 2015, 15(15), 3147-3153; Chang, L. et al. Nanoscale 2016, 8(1), 243-252; Chang, L. et al. Small 2016, 12(43), 5971-5980; and Ding, X. et al. Nat Biomed Eng 2017, 1). Efficient transfection of K-562 and primary human T cells using the Jurkat-optimized DMP protocol was also observed (49% & 82% yields, respectively), thus demonstrating the versatility and potential clinical relevance of the DMP device concept. However, the lower transfection yields relative to that of the Jurkats suggests opportunity for further improvement. We envision potential for doing so through refinement of the device operational parameters to accommodate any differences in the structure or injury response that may lie between the Jurkats and the other cell types.

While further studies are required to elucidate the mechanisms underlying the high transfection yields observed herein for DMP, recent reports from other microfluidic intracellular delivery device development efforts may provide preliminary insights in this regard. For example, as discussed previously, high viability may result from the limitation of poration to a single site in the plasma membrane, which minimizes cellular damage (Boukany, P. E. et al. Nat Nanotechnol 2011, 6(11), 747-754 and Chang, L. et al. Small 2015, 11 (15), 1818-1828). Additionally, high transfection efficiency may result from the opportunity provided for direct cytosolic delivery, which reduces potential for trapping of the exogenous cargo within endosomal or lysosomal vesicles (Sharei, A. et al. Proceedings of the National Academy of Sciences 2013, 110(6), 2082-2087). Finally, since the Penetrator length is comparable to the cell radius, this suggests potential for mechanical disruption of the nuclear envelope as well. Such disruption would be expected to facilitate intra-nuclear delivery, and thus, reduce potential for construct degradation within the cytosol (Ding, X. et al. Nat Biomed Eng 2017, 1).

As discussed earlier, the limitations of conventional viral and non-viral techniques for cellular engineering are well-known. Consequently, this presents wide-ranging opportunity for new non-viral techniques that can safely and efficiently introduce exogenous cargo into cells, particularly difficult-to-transfect cells such as T cells, and do so in a manner that is compatible with prevailing high-volume engineered cell product manufacturing schemes. Emerging CAR T therapies represent one compelling example in this regard, since this could enable circumvention of the looming manufacturing roadblock imposed by the current reliance upon viral transduction, which may limit the potential for extending these promising therapies beyond hematologic malignancies to the far larger population of patients with solid tumors (Maus, M. V. et al. Blood 2014, 123(17), 2625-2635). Non-viral transfection may also provide a safer and more economical means for evaluating new tumor antigen targets relative to viral transduction (Zhao, Y. et al. Cancer Res 2010, 70(22), 9053-9061), thus addressing another critical roadblock to the eventual extension of CAR T therapies to solid tumor indications (Maus, M. V. et al. Blood 2014, 123(17), 2625-2635). While further studies are required to determine whether DMP can address these specific needs, the flexibility of this approach, combined with the encouraging data reported herein, begins to suggest promise in this regard.

In conclusion, we have reported a new microfluidic device concept for transfecting suspension cells that is specifically designed to meet the needs of engineered cell product manufacturing. The novelty of the concept lies in the opportunity it provides for deterministically porating large numbers of cells, each at a single site in their plasma membrane and optional nuclear membrane penetration, and doing so in a manner that allows rapid collection of the cells for subsequent processing. Using human primary cells and cell lines of direct relevance to ex vivo cell therapies, including immune cells that are typically refractory to transfection, we show that DMP enables efficient delivery of large-molecule cargos while minimizing damage to the cell, thus allowing achievement of transfection yields that exceed both conventional and emerging non-viral transfection techniques. This, therefore, suggests that DMP may provide new means for addressing critical roadblocks in the development and manufacture of ex vivo cell therapies based on engineered T cells (e.g., CAR T cell cancer immunotherapies). Moreover, given the inherent versatility of the DMP concept, we envision opportunity for its eventual extension to a wide variety of other applications where progress is currently being hampered by the limitations of existing cellular engineering techniques.

### Materials and Methods

Device Fabrication. The DMP devices were fabricated using 100 mm diameter silicon-on-insulator substrates with 20 μm device, 2 μm buried SiO₂ (BOX), and 500 μm handle layers (Ultrasil Corporation). Front and backside SiO₂ etch masks with 1 μm & 2 μm thicknesses, respectively, were first deposited using a combination of wet oxidation (CVD Equipment Corp; 7/4 sccm H₂/O₂ & 1000° C.) and plasma enhanced chemical vapor deposition (Plasmatherm 790, Unaxis: 400/900 sccm 2% SiH₄/N₂O, 900 mT, & 25 W). The frontside Aspiration Via oxide mask was then patterned by projection lithography (GCA 6300, RTC) and fluorine-based reactive ion etching (RIE) (Multiplex RIE, STS: 30/20 sccm CHF₃/CF₄, 100 mT, & 300 W). This was followed by Backside Aspiration Port oxide mask patterning using contact lithography (MA-6, Suss Microtec) and fluorine-based RIE. The Backside Aspiration Port was then defined using Si deep reactive ion etching (DRIE) (MESC ICP, STS: Etch cycle—130/13 sccm SF₆/O₂, 37 mT, 700/20 W source/platen, & 14 s; Passivation cycle—85 sccm C4F8, 24 mT, 600/0 W source/platen, & 7 s). Afterwards, the Capture Wells and Penetrators were simultaneously defined using frontside isotropic Si RIE (MESC ICP: 95/13 sccm SF₆/O₂, 12 mT, & 500/20 W source/platen). The Aspiration Vias were then defined by frontside Si DRIE using the Aspiration Via oxide mask as a shadow-mask (MESC ICP: Etch cycle—130/13 sccm SF₆/O₂, 24 mT, 600/17 W source/platen, & 7 s; Passivation cycle—85 sccm C₄F₈, 14 mT, 600/0 W source/platen, & 5 s). Finally, the residual frontside oxide mask was removed using fluorine-based RIE, followed by Penetrator tip refinement by chlorine-based RIE (E640, Panasonic Factory Solutions: 10 sccm Cl₂, 1.2 Pa, & 400/12 W source/platen) and fluorine-based RIE (Multiplex RIE: 40/50 sccm O₂/CF₄, 100 mT, & 300 W), and lastly, BOX layer removal by backside fluorine-based RIE (Multiplex RIE: 30/20 sccm CHF₃/CF₄, 100 mT, & 300 W). Scanning electron microscopy was used throughout for fabrication process characterization and device feature verification (Leo Supra 55, Zeiss).

Device Packaging and Experimental Apparatus. The device package was fabricated from polycarbonate and designed for placement on the stage of an upright fluorescence microscope (BX50, Olympus). The microscope was equipped with a CCD camera (Retiga EXi, Q Imaging) and high-intensity lamp (Sola Light Engine, Lumencor), which enabled real-time visualization during device operation. A programmable syringe pump (PhD 2000, Harvard Apparatus) was used for bidirectional actuation of the aspiration circuit.

Cell Culture. The Jurkat and K-562 cells (ATCC) were each cultured at 37° C. and 5% CO₂ in RPMI medium (1640, Lonza), with 10% fetal bovine serum (FBS, Hyclone Labs). The media was refreshed every 48 h, and the cultures were passaged at population densities of 10⁶ cells/mL (MUSE, EMD Millipore). Primary T cell culture was derived from human donor Tn/mem population and maintained under 37° C. and 5% CO₂ in X-Vivol5 media (BioWhittaker) containing 10% fetal calf serum (HyClone, GE Healthcare), supplemented with 50 U/mL recombinant human (rh) IL-2, and 0.5 ng/mL rhIL-15 (Brown, C. E. et al. Molecular Therapy 2018, 26(1), 31-44). Cells were stimulated with Dynabeads Human T Expander CD3/CD28 (Invitrogen) for 7 d and expanded out to days 18-25 for experiments.

Capture Optimization. Prior to their introduction into the DMP device, the Jurkats were stained with 10 μM Calcein Blue AM (CBAM, Life Technologies) in phosphate-buffered saline (PBS, Life Technologies) for 30 min. The cells were then centrifuged and resuspended in fresh PBS with 4% glycerol (to enhance cell settling during the capture process). The device was infused with fresh PBS in an amount sufficient to partially fill the reservoir, and the CBAM-stained cells were pipetted into the reservoir, followed by actuation of the aspiration circuit using the syringe pump. Pipetting was then used to wash away uncaptured cells from the device, and subsequently remove them from the reservoir. Finally, the fluorescence microscope was used to build a photomosaic of the captured cells across the entirety of the device array. Capture Site occupancy (i.e., % of occupied Capture Sites) was determined using image analysis software (Image J, NIH), coupled with a machine learning segmentation plugin (WEKA, University of Waikato, New Zealand). The analysis parameters were defined to exclude both cellular debris and cellular aggregates.

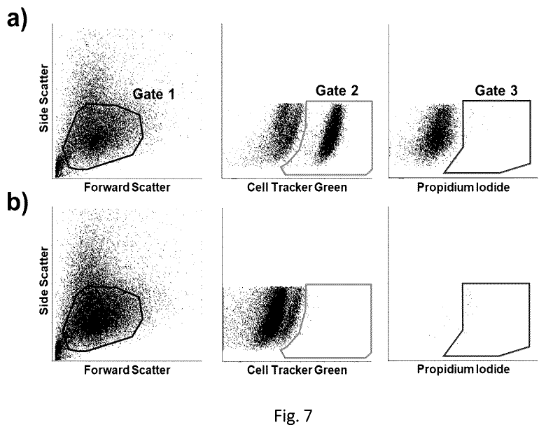

Puncture Optimization. The device was first infused with 0.1 μg/mL propidium iodide (PI, Sigma-Aldrich) in PBS. CBAM-stained Jurkats in 4% glycerol/PBS were then pipetted into the reservoir, captured at 30 μL/min, and punctured at flow rates ranging from 20 μL/min to 60 μL/min. Uncaptured cells were then removed from the device by pipette-based washing, followed by reversal of the aspiration flow to release the captured cells. The released cells were then collected from the reservoir, and co-incubated with 0.1 μg/mL PI and 1 μM CellTracker Green CMFDA Dye (CTG, Life Technologies) for 30 min. Finally, the cells were centrifuged, resuspended in FACS stain solution (Gibco), and assayed using flow cytometry (MACSQuant Analyzer, Miltenyi Biotec). Subsequent analysis of the flow cytometry data was performed using a commercial software package (FCS Express 6, De Novo Software). Using a cascading gating scheme based on control data reported in FIG. 7 in the Supporting Information, cellular viability was defined as the percentage of viable intact cells, relative to the population of intact cells recovered from the device (i.e., Gate 2/Gate 1×100%). Delivery efficiency was defined as the percentage of viable intact cells with exogenous cargo delivered, relative to the population of viable intact cells (i.e., Gate 3/Gate 2×100%). Finally, delivery yield was defined as the percentage of viable intact cells with exogenous cargo delivered, relative to the population of intact cells (i.e., Gate 3/Gate 1×100, which is equivalent to the product of cellular viability and delivery efficiency).

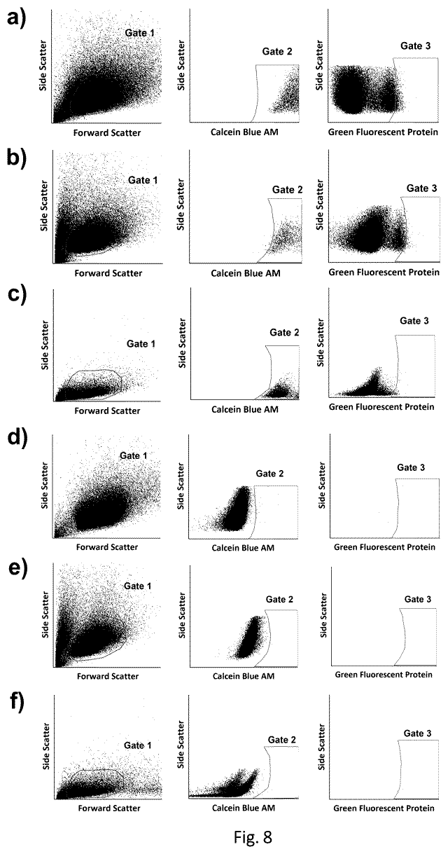

DMP Validation. The device was first infused with 20 μg/mL GFP plasmid in PBS. Jurkat, K-562, or primary human T cells previously stained using 4 μM CellTrace Calcein Red-Orange AM (ThermoFisher), and resuspended in 4% glycerol/PBS, were then introduced into the device, captured at 30 μL/min, and punctured at 40 μL/min. Uncaptured cells were then removed from the device by pipette-based washing, followed by reversal of the aspiration flow to release the captured cells. The released cells were then collected from the reservoir, and incubated in GFP plasmid/PBS for 30 min. Afterwards, they were centrifuged, resuspended in fresh RPMI with 0.5% Penicillin-Streptomycin, and incubated in a humidified 37° C./5% CO₂ incubator for 12 h. Finally, the cells were centrifuged, stained with 8 μM CBAM in PBS for 30 min, followed by centrifugation and resuspension in FACS stain for flow cytometry. Cellular viability, transfection efficiency, and transfection yield were defined in a similar manner as the puncture optimization studies (with gating based on control data reported in FIG. 8).

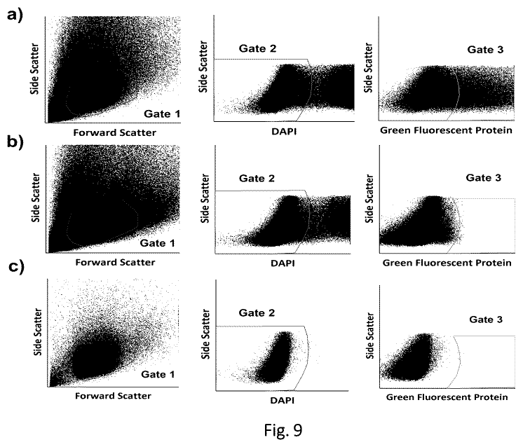

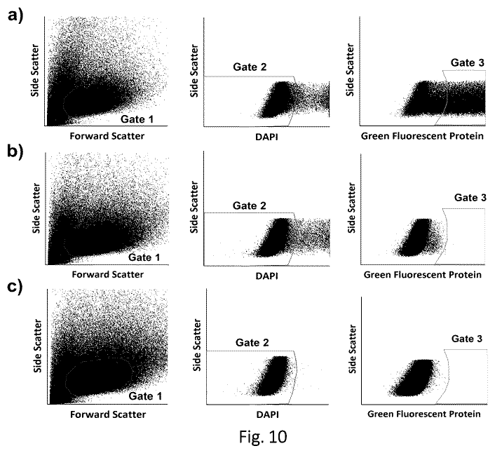

BEP Benchmarking. The benchmarking studies were performed using a commercial BEP instrument (Nucleofector 1, Lonza), with manufacturer-recommended protocols and plasmid concentrations for Jurkats (i.e., Cell Line Nucleofector Kit V, Program X-01, and 20 μg/mL GFP plasmid), K-562 cells (i.e., Cell Line Nucleofector Kit V, Program T-03, and 20 μg/mL GFP plasmid), and primary human T cells (i.e., Human T Cell Nucleofector Kit, Program U-14, and 20 μg/mL GFP plasmid). Afterwards, the cells were collected from the instrument, resuspended in fresh media with 0.5% Penicillin-Streptomycin, and incubated in a humidified 37° C./5% CO₂ incubator for 12 h. Finally, the cells were centrifuged, stained with 0.80 μM 4,6-diamidino-2-phenylindole (DAPI, ThermoFisher) in PBS for 30 min, followed by centrifugation and resuspension in FACS stain for flow cytometry. Cellular viability, transfection efficiency, and transfection yield were defined in the same manner as the DMP validation studies (with gating based on control data reported in FIGS. 9, 10, & 11 for Jurkat, K-562, & primary human T cells, respectively).

Statistical Analyses. All cell studies were repeated in triplicate and statistical analyses were performed using the student's t-test with two-tailed distribution and two-sample unequal variance (Excel, Microsoft).

We have developed methods that permit mass-producing engineered cells at lower cost for lifesaving therapies using novel microfluidic device technology for gene delivery. The DMP technology uses fluid flow to pull each cell in a large population onto its own tiny needle. The flow is then reversed to release the cells from the needles, leaving a singular and precisely defined pore within each cell that allows for gene delivery. This simple, but elegant nanomechanical poration approach provides significant advantages relative to existing gene delivery techniques. For example, since viral vectors make up a large fraction of the overall manufacturing cost of current cell therapies, their elimination through the use of DMP holds potential for considerable cost reduction.

DMP's unique single-site poration mechanism minimizes damage to the cell, while producing a well-defined pathway for introducing genes. This provides the opportunity for achieving both high delivery efficiency and cellular viability, which is difficult to achieve using other non-viral delivery techniques, such as electroporation. We show that DMP can engineer primary human T cells, as used in CAR-T therapies and T cell receptor (TCR) therapies, with efficiencies that exceed state-of-the-art electroporation by more than four-fold.

The DMP technology provides a basis for engineering ex vivo cell and gene therapies for cancer specifically, as well as genetic disorders and degenerative diseases more broadly.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

All figures, tables, and appendices, as well as patents, applications, and publications, referred to above, are hereby incorporated by reference.

Some embodiments have been described in connection with the accompanying drawing. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

What is claimed is:

1. A method of intracellular delivery of a genetic construct to immune cells comprising:
obtaining a deterministic mechanoporation (DMP) platform that comprises a substrate having a surface and a plurality of capture sites, each said capture site having a boundary shape at the surface adapted and configured to support thereon a cell, and each said capture site having a bottom and including a sub-micron-scale projection extending from the bottom toward the surface of the substrate, wherein said projection is adapted and configured to penetrate a cell membrane and/or wall of the cell, and wherein the substrate has a plurality of aspiration vias situated at the bottom of the capture sites;
introducing the cells to the surface in a liquid media;
capturing the cells within the capture sites by applying a first hydrodynamic force;
applying a second hydrodynamic force on the captured cell and locally rupturing the membrane and/or wall of the cell with the projection,
introducing the genetic construct into the cells, and
releasing the porated cells from the capture sites.

2. The method of claim 1, wherein the immune cells are T cells.

3. The method according to claim 2, wherein the T cells are primary human T cells.

4. The method according to claim 1, wherein a mean transfection yield of the immune cells is from 20-100%.

5. The method according to claim 1, wherein a transfection yield of the immune cells is 2-fold to 20-fold higher than a transfection yield obtainable by bulk electroporation.

6. The method according to claim 1, wherein the genetic construct encodes a chimeric antigen receptor (CAR) that recognizes a specific antigen, wherein the CAR comprises an extracellular antigen recognition domain, a transmembrane domain and a cytoplasmic signaling domain that stimulates the immune cells to target and attack cells expressing the specific antigen.

7. The method according to claim 1, wherein the genetic construct encodes a T cell receptor (TCR) that recognizes a specific peptide displayed in the context of MHC II molecules, wherein the TCR is involved in a pathway that stimulates immune cells to target and attack cells expressing an antigen that contains the specific peptide.

8. The method according to claim 1, wherein the substrate of the DMP platform has greater than 10⁶ capture sites.

9. The method according to claim 1, wherein a flow rate of the liquid media containing the cells is adjusted to a first flow rate during capture to achieve a capture site occupancy of at least 50%.

10. The method according to claim 9, wherein a second flow rate of the liquid media containing the cells during application of the second hydrodynamic force is different compared to the first flow rate during capture.

11. The method according to claim 1, wherein a mean transfection yield of the cells is between 50-100%.

12. The method according to claim 1, wherein a mean transfection yield of the cells is between 75-100%.

13. The method according to claim 1, wherein a mean transfection yield of the cells is between 90-100%.

14. The method of claim 1, wherein the projection has a length comparable to the radius of the cells.

15. The method of claim 14, wherein the genetic construct is introduced into the nucleus of the cells.

16. A method of chimeric antigen receptor (CAR) T cell adoptive immunotherapy in a patient comprising:
obtaining T cells transfected with a chimeric antigen receptor (CAR) gene by the method of claim 1, wherein the CAR recognizes a cell surface antigen of a tumor cell, and
administering the cells to the patient.

17. The method according to claim 16, wherein the patient is treated for a disease selected from the group consisting of a cancer, an infection, human immunodeficiency virus (HIV) infection, transplant rejection and autoimmunity.

18. The method according to claim 16, wherein the patient is treated for a cancer.

19. The method according to claim 18, wherein the cancer is a non-blood or non-hematopoietic based cancer, a solid tumor forming or infiltrating/metastatic cancer, or a cancer selected from the group consisting of a bone cancer, an endocrine cancer, a germ cell cancer, a kidney cancer, a liver cancer, a neuroblastoma and a soft tissue cancer.

20. The method of claim 16, wherein the T cells are autologous to the patient.

21. The method of claim 20, wherein the autologous T cells are administered intravenously as a bolus dose.

22. The method of claim 16, wherein the T cells are allogenic to the patient.

23. The method of claim 22 wherein the allogenic T cells are administered intravenously as a bolus dose.

24. A method of T cell receptor (TCR) therapy in a subject comprising:
obtaining T cells transfected with a T cell receptor gene by the method of claim 1, wherein the T cell receptor recognizes a specific peptide displayed in the context of MHC II molecules on antigen presenting cells, and
administering the cells to the subject.

25. The method according to claim 24, wherein the patient is treated for a disease selected from the group consisting of a cancer, an infection, human immunodeficiency virus (HIV) infection, transplant rejection and autoimmunity.
